(19) United States
(12) Patent Application Publication
(10) Pub. No.: US 2016/0368865 A1
(43) Pub. Date: Dec. 22, 2016

(54) GEMINI SURFACTANTS AND METHODS FOR THEIR PREPARATION AND USE

(71) Applicant: EMPIRE TECHNOLOGY DEVELOPMENT LLC, Wilmington, DE (US)

(72) Inventors: Georgius Abidal ADAM, Edensor Park, New South Wales (AU); William Brenden CARLSON, Seattle, WA (US); Angele SJONG, Louisville, CO (US); Feng WAN, Issaquah, WA (US); Timothy Martin LONDERGAN, Seattle, WA (US)

(73) Assignee: EMPIRE TECHNOLOGY DEVELOPMENT LLC, Wilmington, DE (US)

(21) Appl. No.: 15/101,394

(22) PCT Filed: Dec. 2, 2013
(86) PCT No.: PCT/US13/72593

§ 371 Date: Jun. 2, 2016

(51) Int. Cl. C07C 275/62; C09D 7/12; C09D 133/12

(52) U.S. Cl. CPC C07C 275/62; C09D 133/12; C09D 7/1233; C08K 5/21

(57) ABSTRACT

Disclosed herein are gemini surfactants, and methods for making and using these gemini surfactants. These gemini surfactants may be incorporated in paints and coatings to provide hydrophilic and/or self-cleaning properties.

### BACKGROUND

Coatings and paints are routinely used to beautify and protect substrates. The most simple coatings and paints are made of a polymer (the binder) in a solvent (the vehicle), which is commonly called a lacquer. Paints and coatings modify the appearance of an object by adding color, gloss, or texture, and by blending with or differentiating from a surrounding environment. For example, a surface that is highly light scattering can be made glossy by the application of a paint that has additives that result in a high gloss effect. Conversely, a glossy surface can be made to appear flat by additives. Thus, the painted surface is hidden, altered, and ultimately changed in some manner by the presence of the coating. In addition, paints also protect the surface from the surrounding elements and prevent or reduce the corrosive process.

Paints and coatings, while protecting the substrate from the environment, can become dirty over time. Dirt can dull the coating by increasing light scattering or by modifying the color component of the coating. Dirt can also affect the durability of a paint or coating. Thus, it is desirable to have coatings with a hydrophilic surface and self-cleaning properties. A hydrophilic surface allows water to spread out in a thin layer, thus sweeping dirt off the surface as the water thins out and trickles away. This type of “self-cleaning” behavior is advantageous to an exterior paint, as it keeps the coating clean without requiring extensive cleaning.

### SUMMARY

The current disclosure is directed to paints and coatings with gemini surfactants. In one embodiment, a compound is of formula I:

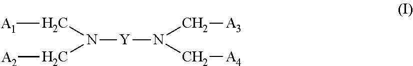

wherein A₁ is —N(CH₂—CH₂—OH)₂, —O—C(═O)—(Z)ₙ—CH₃, —N(—CH₃)—(Z)ₙ—CH₃, —O—C(═O)—CH₂—CH₂—COOH, —O—C(═O)—CH₂—COOH, —O—SO₃H, or —O—PO₃H₂, or salts thereof, where each n is, independently, an integer from 1 to 25, and where Z is alkylene, arylene, alkenylene, alkynylene, acrylene, styrylene, or any combination thereof;

A₂ is —N(CH₂—CH₂—OH)₂, —O—C(═O)—(Z)ₚ—CH₃, —N(—CH₃)—(Z)ₚ—CH₃, —O—C(═O)—CH₂—CH₂—COOH, —O—C(═O)—CH₂—COOH, —O—SO₃H, or —O—PO₃H₂, or salts thereof, where each p is, independently, an integer from 1 to 25, and where Z is alkylene, arylene, alkenylene, alkynylene, acrylene, styrylene, or any combination thereof;

A₃ is —N(CH₂—CH₂—OH)₂, —O—C(═O)—(Z)_{q}—CH₃, —N(—CH₃)—(Z)_{q}—CH₃, —O—C(═O)—CH₂—CH₂—COOH, —O—C(═O)—CH₂—COOH, —O—SO₃H, or —O—PO₃H₂, or salts thereof, where each q is, independently, an integer from 1 to 25, and where Z is alkylene, arylene, alkenylene, alkynylene, acrylene, styrylene, or any combination thereof;

A₄ is —N(CH₂—CH₂—OH)₂, —O—C(═O)—(Z)ᵣ—CH₃, —N(—CH₃)—(Z)ᵣ—CH₃, —O—C(═O)—CH₂—CH₂—COOH, —O—C(═O)—CH₂—COOH, —O—SO₃H, or —O—PO₃H₂, or salts thereof, where each r is, independently, an integer from 1 to 25, and where Z is alkylene, arylene, alkenylene, alkynylene, acrylene, styrylene, or any combination thereof; and

Y is —C(═O)—, —CH₂—CH₂—, —CH₂—(CH₂)ₖ—CH₂—, —C(═O)—NH—C(═O)—, or polyurea, where k is an integer from 1 to 10.

In an additional embodiment, a method of making a surfactant may comprise: contacting any one of urea, biuret, or alkylene diamine with formaldehyde to form a tetrahydroxy methyl compound; contacting the tetrahydroxy methyl compound with any one of fatty acid, anhydride, acid chloride, or a N-methylamino compound to form a di-substituted intermediate compound; and contacting the di-substituted intermediate compound with any one of an amine, succinic anhydride, chlorosulfonic acid, or chlorophosphoric acid to form the surfactant.

In another embodiment, a hydrophilic coating composition may include a surfactant of formula I. In some embodiments, the surfactant may be covalently attached to a binder. In some embodiments, the surfactants may be cross-linked to each other.

In a further embodiment, a method of coating a substrate may include applying a coating composition to the substrate, wherein the coating composition comprises a surfactant of formula I.

### DETAILED DESCRIPTION

This disclosure is not limited to the particular systems, devices and methods described, as these may vary. The terminology used in the description is for the purpose of describing the particular versions or embodiments only, and is not intended to limit the scope.

“Alkylene” refers to a bivalent alkyl moiety having the general formula —(CH₂)ₙ—, where n is from about 1 to about 25, about 1 to about 20, or about 4 to about 20. By bivalent, it is meant that the group has two open sites each of which bonds to another group. Non-limiting examples include methylene, ethylene, trimethylene, pentamethylene, and hexamethylene. Alkylene groups can be substituted or unsubstituted, linear or branched bivalent alkyl groups.

“Alkyl” means a saturated hydrocarbon group which is straight-chained or branched. An alkyl group can contain from 1 to 20 carbon atoms, from 2 to 20 carbon atoms, from 1 to 10 carbon atoms, from 2 to 10 carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, 4,4 dimethylpentyl, octyl, 2,2,4-trimethylpentyl, nonyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

“Substituted alkyl” refers to an alkyl as just described in which one or more hydrogen atoms attached to carbon of the alkyl is replaced by another group, such as halogen, aryl, substituted aryl, cycloalkyl, substituted cycloalkyl, and combinations thereof. Suitable substituted alkyls include, for example, benzyl and trifluoromethyl.

“Alkenylene” refers to a divalent alkenyl moiety, meaning the alkenyl moiety is attached to the rest of the molecule at two positions.

“Alkenyl” means a straight or branched alkyl group having one or more double carbon-carbon bonds and 2-20 carbon atoms, including, but not limited to, ethenyl, 1-propenyl, 2-propenyl, 2-methyl-1-propenyl, 1-butenyl, 2-butenyl, and the like. In some embodiments, the alkenyl chain is from 2 to 10 carbon atoms in length, from 2 to 8 carbon atoms in length, from 2 to 6 carbon atoms in length, or from 2 to 4 carbon atoms in length.

“Alkynylene” refers to a divalent alkynyl moiety, meaning the alkynyl moiety is attached to the rest of the molecule at two positions.

“Alkynyl” means a straight or branched alkyl group having one or more triple carbon-carbon bonds and 2-20 carbon atoms, including, but not limited to, acetylene, 1-propylene, 2-propylene, and the like. In some embodiments, the alkynyl chain is 2 to 10 carbon atoms in length, from 2 to 8 carbon atoms in length, from 2 to 6 carbon atoms in length, or from 2 to 4 carbon atoms in length.

“Arylene” means a bivalent aryl group that links one group to another group in a molecule. Arylene groups may be substituted or unsubstituted.

“Acrylene” means a bivalent acryloyl group that links one group to another group in a molecule. Acrylene groups may be substituted or unsubstituted.

“Styrylene” means a bivalent styryl group that links one group to another group in a molecule. Styrylene groups may be substituted or unsubstituted.

Surfactants are compounds composed of both hydrophilic and hydrophobic or lipophilic groups. In view of their dual hydrophilic and hydrophobic nature, surfactants tend to concentrate at the interfaces of aqueous mixtures; the hydrophilic part of the surfactant orients itself towards the aqueous phase and the hydrophobic part orients itself away from the aqueous phase. Due to these properties, surfactants are generally used as emulsifiers for emulsion polymerization reactions during manufacture of paints. Surfactants, in addition, improve wetting of the substrate by the coating, and wetting of the pigment by the resin. Presence of the surfactants can also affect the mechanical, chemical, freezing, and storage stability of the polymers in paints and emulsions. Additionally, surfactants may also affect the water, moisture, and heat resistance, and adhesiveness of a polymer film. As such, both ionic and non-ionic surfactants may be used in coating compositions.

Gemini surfactants (sometimes called dimeric surfactants) are a new class of surfactants that have two hydrophilic groups and two hydrophobic groups in the molecules. Typically, gemini surfactants have low critical micelle concentrations, and may be used in lower amounts than conventional surfactants. Gemini surfactants can be ten to a thousand times more surface-active than conventional surfactants with similar but single, hydrophilic and hydrophobic groups in the molecules. Further, gemini surfactants may be anionic, cationic, nonionic or amphoteric.

Disclosed herein are gemini surfactants, and methods of making such surfactants. These gemini surfactants may be used in coating compositions and emulsions to provide hydrophilic, self-cleaning properties when applied on a surface.

In some embodiments, a compound is of formula I

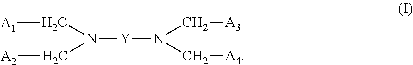

In some embodiments, A₁ may be —N(CH₂—CH₂—OH)₂, —O—C(═O)—(Z)ₙ—CH₃, —N(—CH₃)—(Z)ₙ—CH₃, —O—C(═O)—CH₂—CH₂—COOH, —O—C(═O)—CH₂—COOH, —O—SO₃H, or —O—PO₃H₂, or salts thereof, where each n is, independently, an integer from 1 to 25, and where Z is alkylene, arylene, alkenylene, alkynylene, acrylene, styrylene, or any combination thereof In some embodiments, A₁ may be —N(CH₂—CH₂—OH)₂, —O—C(═O)—(Z)ₙ—CH₃, —N(—CH₃)—(Z)ₙ—CH₃, —O—C(═O)—CH₂—CH₂—COOH, or —O—SO₃H, or salts thereof. In some embodiments, A₁ may be —N(—CH₃)—(CH₂)₂₀—CH₃ or —O—PO₃H₂.

In some embodiments, A₂ may be —N(CH₂—CH₂—OH)₂, —O—C(═O)—(Z)ₚ—CH₃, —N(—CH₃)—(Z)ₚ—CH₃, —O—C(═O)—CH₂—CH₂—COOH, —O—C(═O)—CH₂—COOH, —O—SO₃H, or —O—PO₃H₂, or salts thereof, where each p is, independently, an integer from 1 to 25, and where Z is alkylene, arylene, alkenylene, alkynylene, acrylene, styrylene, or any combination thereof In some embodiments, A₂ may be —N(CH₂—CH₂—OH)₂, —O—C(═O)—(Z)ₚ—CH₃, —N(—CH₃)—(Z)ₚ—CH₃, —O—C(═O)—CH₂—CH₂—COOH, or —O—SO₃H, or salts thereof In some embodiments, A₂ may be —N(—CH₃)—(CH₂)₂₀—CH₃ or —O—PO₃H₂.

In some embodiments, A₃ may be —N(CH₂—CH₂—OH)₂, —O—C(═O)—(Z)_{q}—CH₃, —N(—CH₃)—(Z)_{q}—CH₃, —O—C(═O)—CH₂—CH₂—COOH, —O—C(═O)—CH₂—COOH, —O—SO₃H, or —O—PO₃H₂, or salts thereof, where each q is, independently, an integer from 1 to 25, and where Z is alkylene, arylene, alkenylene, alkynylene, acrylene, styrylene, or any combination thereof In some embodiments, A₃ may be —N(CH₂—CH₂—OH)₂, —O—C(═O)—(Z)_{q}—CH₃, —N(—CH₃)—(Z)_{q}—CH₃, —O—C(═O)—CH₂—CH₂—COOH, or —O—SO₃H, or salts thereof. In some embodiments, A₃ may be —N(—CH₃)—(CH₂)₂₀—CH₃ or —O—PO₃H₂. In some embodiments, A₃ may be —N(CH₂—CH₂—O⁻.Na⁺)₂, —O—C(═O)—CH₂—CH₂—COO⁻.Na⁺, —O—SO⁻₃.Na⁺, or —O—PO₃²⁻.(Na⁺)₂.

In some embodiments, A₄ may be —N(CH₂—CH₂—OH)₂, —O—C(═O)—(Z)ᵣ—CH₃, —N(—CH₃)—(Z)ᵣ—CH₃, —O—C(═O)—CH₂—CH₂—COOH, —O—C(═O)—CH₂—COOH, —O—SO₃H, or —O—PO₃H₂, or salts thereof, where each r is, independently, an integer from 1 to 25, and where Z is alkylene, arylene, alkenylene, alkynylene, acrylene, styrylene, or any combination thereof In some embodiments, A₄ may be —N(CH₂—CH₂—OH)₂, —O—C(50 O)—(Z)ᵣ—CH₃, —N(—CH₃)—(Z)ᵣ—CH₃, —O—C(═O)—CH₂—CH₂—COOH, —O—SO₃H, or salts thereof. In some embodiments, A₄ may be —N(—CH₃)—(CH₂)₂₀—CH₃ or —O—PO₃H₂. In some embodiments, A₄ may be —N(CH₂—CH₂—O⁻.Na⁺)₂, —O—C(═O)—CH₂—CH₂—COO⁻.Na⁺, —O—SO⁻₃.Na⁺, or —O—PO₃²⁻.(Na⁺)₂.

In some embodiments, Y is —C(═O)—, —CH₂—CH₂—, —CH₂—(CH₂)ₖ—CH₂—, —C(═O)—NH—C(═O)—, or polyurea, where k is an integer from 1 to 10.

In some embodiments, the compound of formula I may have the following substitutions at each of, independently, A₁, A₂, A₃, A₄, and Y as shown in Table 1:

TABLE 1

| | | | | |
|---|---|---|---|---|
| Y | A₁ | A₂ | A₃ | A₄ |

| | | | | |
|---|---|---|---|---|
| —C(═O)—, | —N(CH₂—CH₂—OH)₂, | —N(CH₂—CH₂—OH)₂, | —N(CH₂—CH₂—OH)₂, | —N(CH₂—CH₂—OH)₂, |
| —CH₂—CH₂—, | —O—C(═O)—(Z)ₙ—CH₃, | —O—C(═O)—(Z)ₚ—CH₃, | —O—C(═O)—(Z)_{q}—CH₃, | —O—C(═O)—(Z)ᵣ—CH₃, |
| —CH₂—(CH₂)ₖ—CH₂—, | —N(—CH₃)—(Z)ₙ—CH₃, | —N(—CH₃)—(Z)ₚ—CH₃, | —N(—CH₃)—(Z)_{q}—CH₃, | —N(—CH₃)—(Z)ᵣ—CH₃, |
| —C(═O)—NH—C(═O)—, | —O—C(═O)—CH₂—CH₂—COOH, | —O—C(═O)—CH₂—CH₂—COOH, | —O—C(═O)—CH₂—CH₂—COOH, | —O—C(═O)—CH₂—CH₂—COOH, |
| or | —O—C(═O)—CH₂—COOH, | —O—C(═O)—CH₂—COOH, | —O—C(═O)—CH₂—COOH, | —O—C(═O)—CH₂—COOH, |
| polyurea, | —O—SO₃H, or | —O—SO₃H, or | —O—SO₃H, or | —O—SO₃H, or |
| where k is an | —O—PO₃H₂, or | —O—PO₃H₂, or | —O—PO₃H₂, or | —O—PO₃H₂, or |
| integer from 1 | salts thereof, | salts thereof, | salts thereof, | salts thereof, |
| to 10. | where each n is, | where each p is, | where each q is, | where each r is, |
| | independently, | independently, | independently, | independently, an |
| | an integer from | an integer from 1 | an integer from 1 | integer from 1 to |
| | 1 to 25. | to 25. | to 25. | 25. |
| —C(═O)—, | —N(CH₂—CH₂—OH)₂, | —N(CH₂—CH₂—OH)₂, | —N(CH₂—CH₂—OH)₂, | —N(CH₂—CH₂—OH)₂, |
| —CH₂—CH₂—, | —O—C(═O)—(Z)ₙ—CH₃, | —O—C(═O)—(Z)ₚ—CH₃, | —O—C(═O)—(Z)_{q}—CH₃, | —O—C(═O)—(Z)ᵣ—CH₃, |
| —C(═O)—NH—O(═O)—, | —N(—CH₃)—(Z)ₙ—CH₃, | —N(—CH₃)—(Z)ₚ—CH₃, | —N(—CH₃)—(Z)_{q}—CH₃, | —N(—CH₃)—(Z)ᵣ—CH₃, |
| or | —O—C(═O)—CH₂—CH₂—COOH, | —O—C(═O)—CH₂—CH₂—COOH, | —O—C(═O)—CH₂—CH₂—COOH, | —O—C(═O)—CH₂—CH₂—COOH, |
| polyurea. | or | or | or | or |
| | —O—SO₃H. | —O—SO₃H. | —O—SO₃H. | —O—SO₃H. |
| —C(═O)— or | —N(—CH₃)—(CH₂)₂₀—CH₃ | —N(—CH₃)—(CH₂)₂₀—CH₃ | —N(—CH₃)—(CH₂)₂₀—CH₃ | —N(—CH₃)—(CH₂)₂₀—CH₃ |
| —CH₂—CH₂— | or | or | or | or |
| | —O—PO₃H₂ | —O—PO₃H₂ | —O—PO₃H₂ | —O—PO₃H₂ |
| —C(═O)—NH—C(═O)— | —N(—CH₃)—(CH₂)₂₀—CH₃ | —N(—CH₃)—(CH₂)₂₀—CH₃ | —N(CH₂—CH₂—O⁻•Na⁺)₂ | —N(CH₂—CH₂—O⁻•Na⁺)₂ |
| —C(═O)—NH—C(═O)— | —N(—CH₃)—(CH₂)₂₀—CH₃ | —N(—CH₃)—(CH₂)₂₀—CH₃ | —O—C(═O)—CH₂—CH₂—COO⁻•Na⁺ | —O—C(═O)—CH₂—CH₂—COO⁻•Na⁺ |
| —C(═O)—NH—C(═O)— | —N(—CH₃)—(CH₂)₂₀—CH₃ | —N(—CH₃)—(CH₂)₂₀—CH₃ | —O—SO⁻₃•Na⁺ | —O—SO⁻₃•Na⁺ |
| —C(═O)—NH—C(═O)— | —N(—CH₃)—(CH₂)₂₀—CH₃ | —N(—CH₃)—(CH₂)₂₀—CH₃ | —O—PO₃²⁻•(Na⁺)₂ | —O—PO₃²⁻•(Na⁺)₂ |
| —C(═O)—NH—C(═O)— | —O—C(═O)—(CH₂)₂₀—CH₃ | —O—C(═O)—(CH₂)₂₀—CH₃ | —N(CH₂—CH₂—OH)₂ | —N(CH₂—CH₂—OH)₂ |
| —C(═O)—NH—C(═O)— | —O—C(═O)—(CH₂)₂₀—CH₃ | —O—C(═O)—(CH₂)₂₀—CH₃ | —O—C(═O)—CH₂—CH₂—COO⁻•Na⁺ | —O—C(═O)—CH₂—CH₂—COO⁻•Na⁺ |
| —C(═O)—NH—C(═O)— | —O—C(═O)—(CH₂)₂₀—CH₃ | —O—C(═O)—(CH₂)₂₀—CH₃ | —O—SO⁻₃•Na⁺ | —O—SO⁻₃•Na⁺ |
| —C(═O)—NH—C(═O)— | —O—C(═O)—(CH₂)₂₀—CH₃ | —O—C(═O)—(CH₂)₂₀—CH₃ | —O—PO₃²⁻•(Na⁺)₂ | —O—PO₃²⁻•(Na⁺)₂ |
| —CH₂—CH₂— | —O—C(═O)—(CH₂)₂₀—CH₃ | —O—C(═O)—(CH₂)₂₀—CH₃ | —N(CH₂—CH₂—OH)₂ | —N(CH₂—CH₂—OH)₂ |
| —CH₂—CH₂— | —O—C(═O)—(CH₂)₂₀—CH₃ | —O—C(═O)—(CH₂)₂₀—CH₃ | —O—C(═O)—CH₂—CH₂—COO⁻•Na⁺ | —O—C(═O)—CH₂—CH₂—COO⁻•Na⁺ |
| —CH₂—CH₂— | —O—C(═O)—(CH₂)₂₀—CH₃ | —O—C(═O)—(CH₂)₂₀—CH₃ | —O—SO⁻₃•Na⁺ | —O—SO⁻₃•Na⁺ |
| —CH₂—CH₂— | —O—C(═O)—(CH₂)₂₀—CH₃ | —O—C(═O)—(CH₂)₂₀—CH₃ | —O—PO₃²⁻•(Na⁺)₂ | —O—PO₃²⁻•(Na⁺)₂ |
| —CH₂—CH₂— | —N(—CH₃)—(CH₂)₂₀—CH₃ | —N(—CH₃)—(CH₂)₂₀—CH₃ | —N(CH₂—CH₂—OH)₂ | —N(CH₂—CH₂—OH)₂ |
| —CH₂—CH₂— | —N(—CH₃)—(CH₂)₂₀—CH₃ | —N(—CH₃)—(CH₂)₂₀—CH₃ | —O—C(═O)—CH₂—CH₂—COO⁻•Na⁺ | —O—C(═O)—CH₂—CH₂—COO⁻•Na⁺ |
| —CH₂—CH₂— | —N(—CH₃)—(CH₂)₂₀—CH₃ | —N(—CH₃)—(CH₂)₂₀—CH₃ | —O—SO⁻₃•Na⁺ | —O—SO⁻₃•Na⁺ |
| —CH₂—CH₂— | —N(—CH₃)—(CH₂)₂₀—CH₃ | —N(—CH₃)—(CH₂)₂₀—CH₃ | —O—PO₃²⁻•(Na⁺)₂ | —O—PO₃²⁻•(Na⁺)₂ |
| —C(═O)— | —N(—CH₃)—(CH₂)₂₀—CH₃ | —N(—CH₃)—(CH₂)₂₀—CH₃ | —N(CH₂—CH₂—OH)₂ | —N(CH₂—CH₂—OH)₂ |
| —C(═O)— | —N(—CH₃)—(CH₂)₂₀—CH₃ | —N(—CH₃)—(CH₂)₂₀—CH₃ | —O—C(═O)—CH₂—CH₂—COO⁻•Na⁺ | —O—C(═O)—CH₂—CH₂—COO⁻•Na⁺ |
| —C(═O)— | —N(—CH₃)—(CH₂)₂₀—CH₃ | —N(—CH₃)—(CH₂)₂₀—CH₃ | —O—SO⁻₃•Na⁺ | —O—SO⁻₃•Na⁺ |
| —C(═O)— | —N(—CH₃)—(CH₂)₂₀—CH₃ | —N(—CH₃)—(CH₂)₂₀—CH₃ | —O—PO₃²⁻•(Na⁺)₂ | —O—PO₃²⁻•(Na⁺)₂ |

Examples of compounds represented by formula I include, but are not limited to, the following compounds:

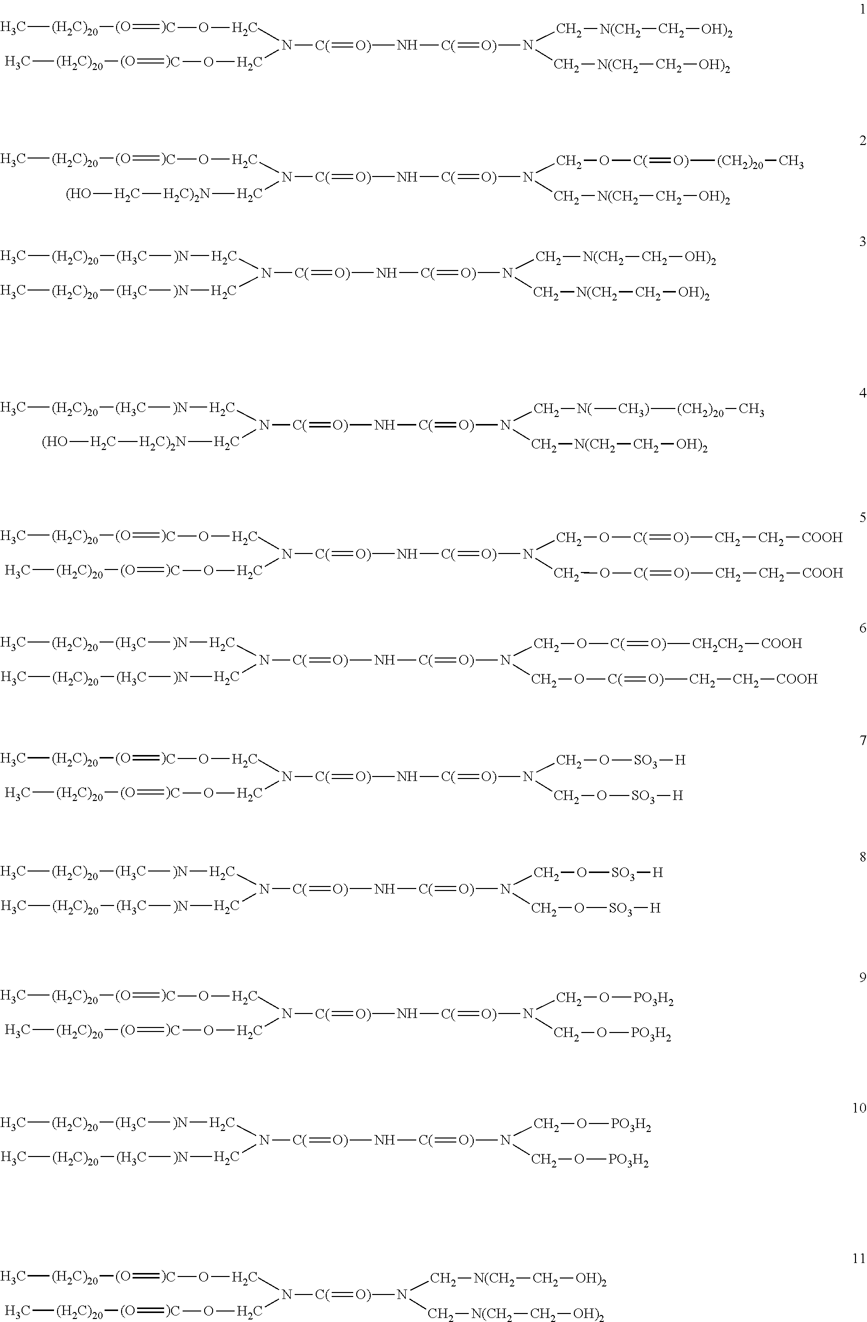

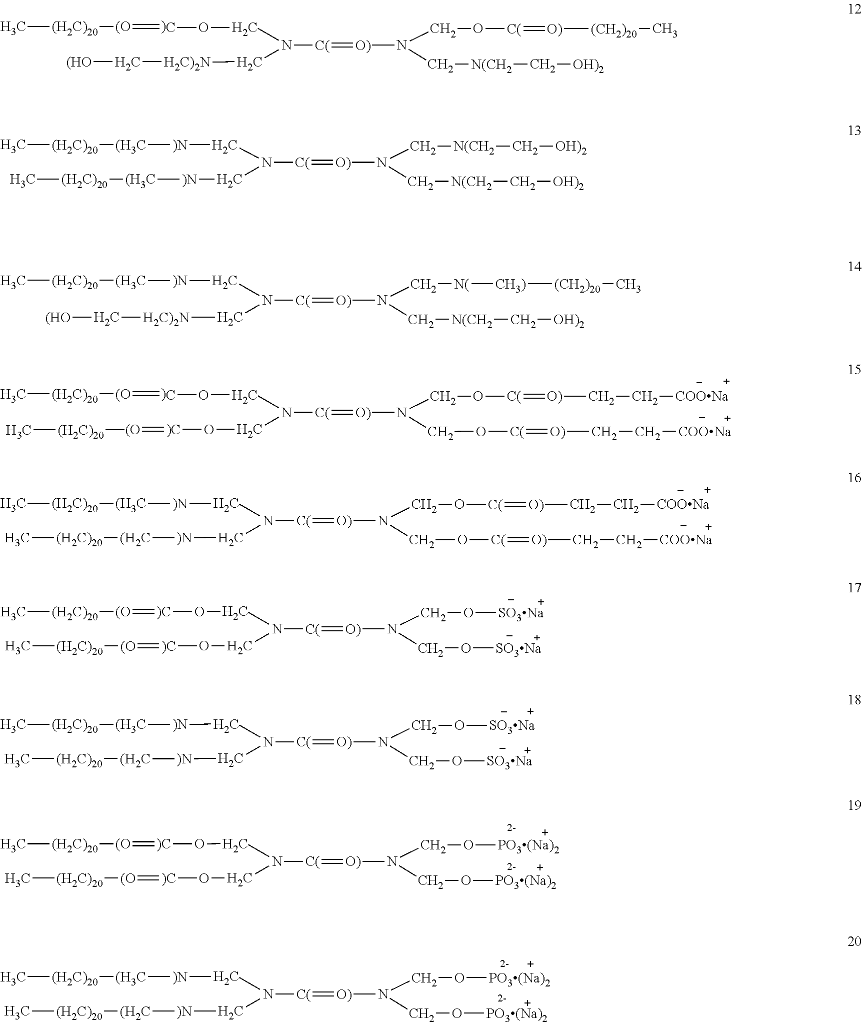

In some embodiments, the compound of formula I may be a gemini surfactant. Gemini surfactants may possess at least two hydrophobic chains and two ionic or polar groups. Gemini surfactants may have a central “spacer” molecule or a group (denoted by —N—Y—N— in formula I) to which the hydrophobic and hydrophilic groups are attached. A great deal of variation may exist in the nature of the spacers. For example, the spacer may be a biuret, urea, alkylene diamine or polyurea. Further, the ionic group may be anionic or cationic. In addition, gemini surfactants may have symmetrical structures with two identical polar groups and two identical hydrophobic groups. In some embodiments, gemini surfactants may be asymmetric. Further, the structure can be adapted to make the surfactant more hydrophobic or more hydrophilic depending on the use. For example, increasing the nonpolar chain length of the hydrophobic groups may increase both the lipophilicity and surface activity, with a decrease in the critical micellar concentration. In some embodiments, the ratio of hydrophobic groups to hydrophilic groups may vary in the gemini surfactants described herein. The ratio of hydrophobic groups to hydrophilic groups may be about 2:2, about 2:1, about 1:2, about 3:1, or about 1:3.

In some embodiments, the hydrophobic groups of the gemini surfactants may be an alkyl ether chain, an arylalkyl ether chain, an alkylester chain, or an arylalkylester chain, with suitable chain length. Such chains can act as anchors and prevent leaching of the surfactants when incorporated in paints. In some embodiments, the hydrophilic groups may be monoethanol amine, diethanol amine, or triethanol amine; anionic groups, such as carboxylate, sulphate, sulphonate, monohydrogen phosphate, or dihydrogen phosphate, or salts of Na⁺, K⁻, Ca²⁺, Mg²⁺, or NH₄⁺, or any combination thereof; cationic groups, such as quaternary ammonium salts, phosphonium salts, acrylate salts, or any combination thereof

In some embodiments, a hydrophilic coating may include a surfactant of formula I, as described herein. The surfactant may be a gemini surfactant, and the hydrophilic coating may provide hydrophilic and/or self-cleaning properties when applied on a substrate. As water evaporates, binder particles pack against each other forming an irreversible networked structure. During this process, coalescing agents along with gemini surfactants may migrate to the surface. The gemini surfactant may provide a hydrophilic surface to the coating, thus aiding in self-cleaning of the surface. These surfaces are able to interact and retain water molecules for relatively longer periods of time, thus keeping the surface wet and helping water to sheathe off and remove dirt. In addition, the quaternary ammonium salt surfactants may provide anti-bacterial and anti-microbial properties to the coating.

Gemini surfactant may be present in the coating composition at about 0.5 to about 5 weight percent, at about 0.5 to about 2.5 weight percent, at about 0.5 to about 2 weight percent, at about 0.5 to about 1.5 weight percent, or at about 0.5 to about 1 weight percent. Specific examples include about 0.5 weight percent, about 1 weight percent, about 1.5 weight percent, about 2 weight percent, about 2.5 weight percent, about 5 weight percent of the total weight, and ranges between (and including the endpoints of) any two of these values. Due to the high surface-activity, a much lower concentration of the surfactants may be needed as compared to the conventional surfactants.

Gemini surfactants may be added to the coating during emulsion polymerization process by substituting the conventional surfactants with the gemini surfactants described herein. In an emulsion polymerization process, the surfactant is dissolved in water until the critical micelle concentration (CMC) is reached. The interior of the micelle provides the site necessary for polymerization. The polymerization process involves heating a mixture containing water, an initiator, monomer and a surfactant with constant stirring. The initiator/surfactant mixture and monomer are vigorously mixed to form micelles. In some embodiments, the gemini surfactants may be mixed with conventional surfactants during this process. Examples of conventional surfactants that may be used include, but are not limited to, alkyl phenol ethoxylates, sodium lauryl sulfate, dodecylbenzenesulfonate, polyoxyethylene alkyl ethers, polyoxyethylene alkyl allyl ethers, ethylene glycols, polyoxyethylene, stearic acid and polyoxypropylene. In some embodiments, the gemini surfactants may be incorporated in the paint composition at the end of the process, and mixed with the paint before use. For example, an end consumer may add the gemini surfactant to any conventional paint formulation before use.

In some embodiments, the gemini surfactants in the paint composition may exist as molecules cross-linked to each other. The presence of cross-linking groups, such as acrylene or styrylene groups may be involved in this cross-linking. In some embodiments, the gemini surfactants may exist as free molecules without cross-links. In addition, the gemini surfactants may also exist as cross-linked to the binder component. The binder may be an acrylate, styrenic or a vinyl polymer. Suitable binder polymers may be polymers of alkylacrylate, alkyl methacrylate, allyl methacrylate, acrylic acid, methacrylic acid, acrylamide, 2-hydroxyethyl methacrylate, 2-hydroxypropyl methacrylate, thioethyl methacrylate, vinyl methacrylate, vinyl benzene, 2-hydroxyethyl acrylate, butyl acrylate, 2-ethylhexyl acrylate, vinyltrimethoxysilane, vinyltriethoxysilane, vinyl formate, vinyl acetate, vinyl propionate, vinyl butyrate, vinyl hexanoate, vinyltoluene, α-methyl styrene, chlorostyrene, or styrenesulfonic acid, or a copolymer of any of the foregoing, or any combination thereof.

In some embodiments, the gemini surfactant may be dispersed in inorganic binders. Inorganic binders may include, without limitation, alkali metal polysilicates, such as potassium polysilicate, sodium polysilicate, lithium polysilicate or the like.

Paints and coatings may contain one or more additives or components in their composition. These additives alter properties of the paint, such as shelf life, application and longevity, health and safety. Such additives may be added, for example, during the manufacture of the emulsion polymer or during the formulation of the paint itself. Additives include initiators, rheology modifiers, preservatives, coalescing agents, and the like. Initiators are a source of free radicals to initiate the polymerization process in which monomers polymerize to form the polymers. Coatings may contain a redox system initiator that promotes polymerization at room temperature, such as ferrous salts, thiosulfate salts, or persulfate salts.

Thickeners and rheology modifiers may also be added to coatings to achieve the desired viscosity and flow properties. Thickeners function by forming multiple hydrogen bonds with the acrylic polymers, thereby causing chain entanglement, looping and/or swelling which results in volume restriction. Thickeners, such as cellulose derivatives including hydroxyethyl cellulose, methyl cellulose and carboxymethyl cellulose, may be used in the compositions.

One or more preservatives may be added in the coating compositions in low doses to protect against the growth of micro-organisms. Preservatives, such as methyl benzisothiazolinones, chloromethylisothiazolinones, barium metaborate and 1-(3-chloroallyl)-3,5,7-triaza-1-azoniaadamantane chloride, may be used.

Coalescing agents, such as ester alcohols, benzoate ethers, glycol ethers, glycol ether esters and n-methyl-2-pyrrolidone, may be added to the coating compositions. Coalescing agents are sometimes added to promote film formation under varying atmospheric conditions. Coalescing agents may be slow evaporating solvents with some solubility in the polymer phase. Coalescing agents may also act as a temporary plasticizer, allowing film formation at temperatures below the system's glass transition temperature. After film formation, the coalescing agents may slowly diffuse to the surface and evaporate, thereby increasing the hardness and block resistance of the film.

Coatings may further contain one or more of the following components or additives: solvents, pigments, plasticizers, and the like. One or more plasticizers may be added to the compositions to adjust the tensile properties of the paint film. Plasticizers may be, for example, a glucose-based derivative, a glycerine-based derivative, propylene glycol, ethylene glycol, phthalates and the like.

The paints according to the disclosure may further include hydrophilic agents attached to one or more pigments. The term “pigments” is intended to embrace, without limitation, pigmentary compounds employed as colorants, including white pigments, as well as ingredients commonly known in the art as “opacifying agents” and “fillers”. Pigments may be any particulate organic or inorganic compound and may provide coatings with the ability to obscure a background of contrasting color (hiding power).

The present disclosure describes hydrophilic coating compositions that when applied to a substrate and cured, result in a hydrophilic coating. A hydrophilic coating composition may be a liquid hydrophilic coating composition, such as a solution or a dispersion comprising a liquid medium. Any liquid medium that allows application of the hydrophilic coating formulation on a surface would suffice. Examples of liquid media are alcohols, like methanol, ethanol, propanol, butanol, acetone, methylethyl ketone, tetrahydrofuran, dichloromethane, toluene, and aqueous mixtures or emulsions thereof, or water. The coating compositions may also be a latex emulsion, an aqueous solution, a non-aqueous solution, or a powder. The hydrophilic coating composition may further comprise components that when cured are converted into the hydrophilic coating, and thus remain in the hydrophilic coating after curing. As used herein, curing refers to physical or chemical hardening or solidifying by any method, for example heating, cooling, drying, crystallizing, or curing as a result of a chemical reaction, such as radiation-curing or heat-curing. In the cured state, all or a portion of the components in the hydrophilic coating formulation may be cross-linked forming covalent linkages between all or a portion of the components, for example by using UV or electron beam radiation. In addition, in the cured state, all or a portion of the components may be ionically bonded, bonded by dipole-dipole type interactions, or bonded via Van der Waals forces or hydrogen bonds.

To apply the hydrophilic coating on the substrate, a primer coating may be used in order to provide a binding between the hydrophilic coating and the substrate. In some instances, the primer coating facilitates adhesion of the hydrophilic coating to the substrate. The binding between the primer coating and the hydrophilic coating may occur due to covalent or ionic links, hydrogen bonding, or polymer entanglements. These primer coatings may be solvent-based, water-based (latexes or emulsions) or solvent-free and may comprise linear, branched and/or cross-linked components. Typical primer coatings that could be used may include, for example, polyether sulfones, polyurethanes, polyesters, polyacrylates, polyamides, polyethers, polyolefins and copolymers thereof. The hydrophilic coatings can also be applied on the substrate without a primer.

The coatings may be used as a decorative coating, an industrial coating, a protective coating, a UV-protective coating, a self-cleaning coating, a biocidal coating, or any combination thereof. The coatings may generally be applied to any substrate. The substrate may be an article, an object, a vehicle or a structure. Although no particular limitation is imposed on the substrate to be used in the present disclosure, exemplary substrates include an exterior of a building, vehicles, cars, trucks, bicycles, bridges, airplanes, helicopters, metal railings, fences, glasses, plastics, metals, ceramics, wood, stones, cement, fabric, paper, leather, walls, pipes, vessels, medical devices, and the like. The coating may be applied to a substrate by spraying, dipping, rolling, brushing, or any combination thereof.

In addition to its use in paints, the gemini surfactants may also be used as a defoamer, an emulsifier, a dispersant, a wetting aid, a leveling aid, or a demulsifying agent. Gemini surfactants may also be used in sunscreens, skin-cleansing compositions, dermatology and acne care products (for example, soaps, specialty soaps, liquid hand soaps, shampoos, conditioners, shower gels), household products (for example, dry and liquid laundry detergents, dish soaps, dishwasher detergents, toilet bowl cleaners, upholstery cleaners, glass cleaners, general purpose cleaners, or fabric softeners), hard surface cleaners (for example, floor cleaners, metal cleaners, automobile and other vehicle cleaners), pet care products (for example, shampoos), and cleaning products in general. Other uses for gemini surfactants may be found in industrial applications in lubricants, emulsion polymerization, textile processing, mining flocculates, petroleum recovery, dispersants for pigments, wetting or leveling agents in paints and printing inks, wetting agents for household and agricultural pesticides, wastewater treatment and collection systems, off-line and continuous cleaning, and manufacture of cross-flow membrane filters, such as reverse osmosis (RO), ultra filtration (UF), micro filtration (MF) and nano filtration (UF), plus membrane bioreactors (MBRs), and all types of flow-through filters including multi-media filters, and many other products and processes. Further, the gemini surfactants may also be used as dispersants for tramp oil in cooling towers and after oil spills.

In some embodiments, a method of making a surfactant includes: contacting any one of urea, biuret, or alkylene diamine with formaldehyde to form a tetrahydroxy methyl compound; contacting the tetrahydroxy methyl compound with any one of fatty acid, anhydride, acid chloride or the N-methylamino compound to form a di-substituted intermediate compound; and contacting the di-substituted intermediate compound with any one of an amine, succinic anhydride, chlorosulfonic acid, or chlorophosphoric acid to form the surfactant.

In some embodiments, the urea, biuret, or alkylene diamine with formaldehyde may be contacted in a molar ratio from about 1:2 to about 1:6, from about 1:2 about 1:5, from about 1:2 to about 1:4, or from about 1:2 to about 1:3. Specific examples include, but are not limited to, about 1:2, about 1:3, about 1:4, about 1:5, about 1:6, and ranges between any two of these values. The alkylene diamine may be ethylene diamine. This process may be conducted in the presence of a basic catalyst. Specific examples of the basic catalyst include alkali metal hydroxides, such as KOH, LiOH, NaOH, and the like. Contacting any one of urea, biuret, or alkylene diamine with the formaldehyde and the basic catalyst may be performed in a solution. During this process, the pH of the solution may be maintained from about pH 8 to about pH 11, from about pH 8 to about pH 10.5, from about pH 8 to about pH 10, from about pH 8 to about pH 9, or from about pH 8 to about pH 8.5. Specific examples include, but are not limited to, about pH 8, about pH 8.5, about pH 9, about pH 9.5, about pH 10, about pH 11, and ranges between any two of these values (including their endpoints).

When contacting any one of urea, biuret, or alkylene diamine with the formaldehyde and the basic catalyst, the mixture may be heated to a temperature of about 50° C. to about 90° C., about 50° C. to about 75° C., about 50° C. to about 70° C., or about 50° C. to about 60° C. Specific examples also include, but are not limited to, about 50° C., about 65° C., about 70° C., about 80° C., about 85° C., about 90° C., and ranges between (and including the endpoints of) any two of these values. The heating may be performed for about 2 hours to about 6 hours, about 2 hours to about 5 hours, about 2 hours to about 4 hours, or about 2 hours to about 3 hours. Specific examples include, but are not limited to, about 2 hours, about 3 hours, about 4 hours, about 5 hours, about 6 hours, and ranges between (and including the endpoints of) any two of these values.

In some embodiments, the tetrahydroxy methyl compound may be contacted with any one of fatty acid, anhydride, acid chloride or the N-methylamino compound in a molar ratio from about 1:2 to about 1:4, from about 1:2 about 1:3, or from about 1:2 to about 1:2.5. Specific examples include, but are not limited to, about 1:2, about 1:2.5, about 1:3, about 1:4, and ranges between any two of these values. The fatty acid used in this reaction process may be saturated or unsaturated fatty acids of 5-25 carbon atoms in length comprising alkylene, arylene, alkenylene, alkynylene, acrylene, or styrylene groups, or any combination thereof. In some embodiments, fatty acid chlorides may also be used in place of anhydrides. Examples include, but are not limited to, anhydrides or chlorides of myristic acid, palmitic acid, stearic acid, arachidic acid, cerotic acid, myristoleic acid, palmitoleic acid, oleic acid, linoleic acid, or any other long chain alkanoic acids. Similarly, the N-methylamino compound may be of 5-25 carbon atoms in length. Examples of N-methylamino compounds that may be used in this process include, N-methyl decylamine, N-methyl dodecylamine, N-methyl tridecylamine, N-methyl tetradecylamine, N-methyl pentadecylamine, N-methyl heneicosylamine, or any other long chain N-alkyl amine derivatives.

When contacting the tetrahydroxy methyl compound with any one of fatty acid, anhydride, acid chloride or the N-methylamino compound, the mixture may be reacted at ambient temperature of about 20° C. to about 30° C., about 20° C. to about 28° C., about 20° C. to about 25° C., or about 20° C. to about 22° C. Specific examples also include, but are not limited to, about 20° C., about 22° C., about 25° C., about 28° C., about 30° C., and ranges between (and including the endpoints of) any two of these values. The heating may be performed for about 0.5 hours to about 3 hours, for about 0.5 hours to about 1.5 hours, or for about 0.5 hours to about 1 hour. Specific examples include, but are not limited to, about 0.5 hours, about 1 hour, about 2 hours, about 3 hours, and ranges between (and including the endpoints of) any two of these values.

In some embodiments, the di-substituted intermediate compound is contacted with any one of the amine, succinic anhydride, chlorosulfonic acid, or chlorophosphoric acid in a molar ratio of about 1:3 to about 1:6, about 1:3 to about 1:5, or about 1:3 to about 1:4. Specific examples include about 1:3, about 1:4, about 1:5, about 1:6, and ranges between (and including the endpoints of) any two of these values. The disubstituted compound may be in a solvent, such as ethanol, tetrahydrofuran, or dioxane. The amine may be a trialkyl amine, monoethanol amine, diethanol amine or triethanol amine. The mixture of di-substituted compound and any one of the amine, succinic anhydride, chlorosulfonic acid, or chlorophosphoric acid may be reacted at ambient temperature of about 20° C. to about 30° C., about 20° C. to about 28° C., about 20° C. to about 25° C., or about 20° C. to about 22° C. Specific examples also include, but are not limited to, about 20° C., about 22° C., about 25° C., about 28° C., about 30° C., and ranges between (and including the endpoints of) any two of these values. The heating may be performed for about 1 hour to about 4 hours, for about 1 hour to about 3 hours, for about 1 hour to about 2.5 hours, or for about 1 hour to about 2 hours. Specific examples include, but are not limited to, about 1 hour, about 1.5 hours, about 2 hours, about 3 hours, about 4 hours, and ranges between (and including the endpoints of) any two of these values.

In some embodiments, the surfactant formed by the method described herein may be neutralized with hydroxides, such as NaOH, KOH, NH₄OH, Mg(OH)₂, Ca(OH)₂, or any combination thereof, to obtain salts. The hydrophilic ends of the surfactants comprising carboxylates, sulfates, sulfonates and phosphates may react with such hydroxides to form the respective salts.

### EXAMPLES

### Example 1

### Preparation of gemini surfactant compound 1

About 103 grams of biuret and 324 grams of formalin solution (37 weight % concentration) are mixed in a five-neck flanged top reaction flask fitted with a condenser, mechanical stirrer, dropping funnel, and a thermometer. The reaction is started by adding 100 mL of 40 weight % sodium hydroxide solution drop wise, and adjusting the pH of the reaction mixture to pH 10. The reaction mixture is mixed and heated to about 65° C. for 2 hours, maintaining the pH between pH 9-pH 10. At the end of this period, the reaction mixture is cooled and neutralized with a cold (5-10° C.) solution of sodium dihydrogen phosphate. The product is desalted and dried with molecular sieves. The product is evaporated by rotary evaporators and dried under vacuum to obtain the tetrahydroxy methyl compound.

The flanged top reaction vessel fitted with mechanical stirrer, thermometer, condenser and dropping funnel immersed in water bath is charged with diethanol amine (110 grams). About 106 grams of the tetrahydroxy methyl compound obtained above is dissolved in 150 grams of ethanol and added via a dropping funnel. The reaction is maintained at 30° C. and the tetrahydroxy methyl compound is added dropwise for one hour, with constant mixing. At the end of the addition, the reaction mixture is further mixed for 3 hours. The unreacted product and ethanol are separated by rotary evaporation under vacuum, and the di-substituted compound is obtained.

The above obtained di-substituted compound is dissolved in 100 grams of tetrahydrofuran (THF) and added drop wise and mixed with docosanoic acid chloride at 30° C. The mixture is heated to 50° C. and the reaction is continued for three hours. The mixture is cooled to room temperature and the product is neutralized with 10% sodium bicarbonate. The solvent and water are evaporated, and the fatty acid salt is separated by extraction. The final product is re-dissolved, desalted and dried with molecular sieves. The solvent is evaporated under vacuum to obtain compound 1.

### Example 2

### Preparation of Gemini Surfactant Compound 3

About 103 grams of biuret and 324 grams of formalin solution (37 weight% concentration) are mixed in a five-neck flanged top reaction flask fitted with a condenser, mechanical stirrer, dropping funnel, and a thermometer. The reaction is started by adding 100 mL of 40 weight % sodium hydroxide solution drop wise, and adjusting the pH of the reaction mixture to pH 10. The reaction mixture is mixed and heated to about 65° C. for 2 hours, maintaining the pH between pH 9-pH 10. At the end of this period, the reaction mixture is cooled and neutralized with a cold (5-10° C.) solution of sodium dihydrogen phosphate. The product is desalted and dried with molecular sieves. The product is evaporated by rotary evaporators and dried under vacuum to obtain the tetrahydroxy methyl compound.

The above obtained hydroxyl methyl compound (106.5 grams, 0.5 mole) is dissolved in methanol and added drop wise to one mole of N-methyl heneicosylamine at 30° C. The mixture is maintained at 30° C. with efficient mechanical mixing for further two hours. The product is desalted and dried with molecular sieves. The product is evaporated by rotary evaporators and dried under vacuum to obtain the di-substituted compound.

The di-substituted compound obtained above is dissolved in ethanol and reacted with one mole of diethanol amine (110 grams) at 30° C. with efficient mixing. The reaction mixture is heated to 70° C. for 2 hours. At the end of this period, the mixture is cooled to room temperature, and the product is purified from the unreacted diethanolamine and the solvent by rotary evaporation, then dried under vacuum at 60° C. to obtain compound 3.

### Example 3

### Preparation of Gemini Surfactant Compound 5

About 103 grams of biuret and 324 grams of formalin solution (37 weight % concentration) are mixed in a five-neck reaction flask fitted with a condenser, mechanical stirrer, dropping funnel, and a thermometer. The reaction is started by adding 100 mL of 40 weight% sodium hydroxide solution drop wise, and the pH of the reaction mixture is adjusted to pH 10. The reaction mixture is heated to about 65° C. for 2 hours with efficient mechanical mixing, and the pH is maintained at between pH 9-pH 10. At the end of this period, the reaction mixture is cooled and neutralized with a cold (5-10° C.) solution of sodium dihydrogen phosphate. The product is desalted and dried with molecular sieves. The product is evaporated by rotary evaporators and dried under vacuum to obtain the tetrahydroxy methyl compound.

The above obtained hydroxyl methyl compound (106 grams) is dissolved in THF and added drop wise to two moles of mixture consisting of docosanoic acid chloride and succinic anhydride in a ratio of 1:1, at 30° C. and mixed for two hours. The mixture is heated to 70° C. for further 2 hours and later cooled to room temperature. The product is neutralized with 10% sodium bicarbonate, desalted, and dried with molecular sieves. The product is evaporated by rotary evaporators and dried under vacuum to obtain the gemini surfactant (compound 5).

### Example 4

### Preparation of Gemini Surfactant Compound 7

About 103 grams of biuret and 324 grams of formalin solution (37 weight % concentration) are mixed in a five-neck reaction flask fitted with a condenser, mechanical stirrer, dropping funnel, and a thermometer. The reaction is started by adding 100 mL of 40 weight % sodium hydroxide solution drop wise, and the pH of the reaction mixture is adjusted to pH 10. The reaction mixture is heated to about 65° C. for 2 hours with efficient mechanical mixing, and the pH is maintained at between pH 9-pH 10. At the end of this period, the reaction mixture is cooled and neutralized with a cold (5-10° C.) solution of sodium dihydrogen phosphate. The product is desalted and dried with molecular sieves. The product is evaporated by rotary evaporators and dried under vacuum to obtain the tetrahydroxy methyl compound.

The above obtained hydroxyl methyl compound (106 grams) is dissolved in THF and added drop wise to two moles of mixture consisting of docosanoic acid and chlorosulfonic acid in a ratio of 1:1, at 30° C. and mixed for two hours. The mixture is heated to 70° C. for further 2 hours and later cooled to room temperature. The product is neutralized with 10% sodium bicarbonate, desalted, and dried with molecular sieves. The product is evaporated by rotary evaporators and dried under vacuum to obtain the gemini surfactant (compound 7).

### Example 5

### Preparation of Gemini Surfactant Compound 9

About 103 grams of biuret and 324 grams of formalin solution (37 weight % concentration) are mixed in a five-neck reaction flask fitted with a condenser, mechanical stirrer, dropping funnel, and a thermometer. The reaction is started by adding 100 mL of 40 weight % sodium hydroxide solution drop wise, and the pH of the reaction mixture is adjusted to pH 10. The reaction mixture is heated to about 65° C. for 2 hours with efficient mechanical mixing, and the pH is maintained at between pH 9-pH 10. At the end of this period, the reaction mixture is cooled and neutralized with a cold (5-10° C.) solution of sodium dihydrogen phosphate. The product is desalted and dried with molecular sieves. The product is evaporated by rotary evaporators and dried under vacuum to obtain the tetrahydroxy methyl compound.

The above obtained hydroxyl methyl compound (106 grams) is dissolved in THF and added drop wise to two moles of mixture consisting of docosanoic acid and chlorophosphoric acid in a ratio of 1:1, at 30° C. and mixed for two hours. The mixture is heated to 70° C. for further 2 hours and later cooled to room temperature. The product is neutralized with 10% sodium bicarbonate, desalted, and dried with molecular sieves. The product is evaporated by rotary evaporators and dried under vacuum to obtain the gemini surfactant (compound 9).

### Example 6

### A hydrophilic Paint with Gemini Surfactant

About 10 grams of compound 1 is mixed with 40 grams of TiO₂, 2 grams of thickener (hydroxyethyl cellulose), 150 grams of solvent (water), 70 grams of binder (methyl methacrylate), 0.3 grams of coalescing agent (2,2,4-trimethyl-1,3-pentanediolmono(2-methylpropanoate)), and 0.05 grams of bactericide. The components are mixed under high shear for 30 minutes.

### Example 7

### Evaluation of Hydrophilic Property

The coating preparation of Example 6 is coated on a glass surface and dried at room temperature. The surface free energy and the water droplet contact angle of the hydrophilic coating are measured as follows. A Zisman plotting method is employed for measuring the surface free energy. The surface tension of various concentrations of the aqueous solution of magnesium chloride is plotted along the X-axis, and the contact angle in terms of cos θ is plotted along the Y-axis. A graph with a linear relationship between the two is obtained. The graph is extrapolated such that the surface tension at contact angle 0° is measured and is defined as the surface free energy of the coated glass surface. The surface free energy of the glass surface measured will be 82 milliNewton/meter. The high surface free energy is indicative of the hydrophilic property of the coating.

### Example 8

### Evaluation of Hydrophilic Coating

A hydrophilic coating is prepared as in Example 6 but using compound 3. The coating is coated on a glass substrate and evaluated for the following properties.

Hydrophilicity: The water droplet contact angle in air is measured by using DropMaster 500 (Kyowa Interface Science Co., Ltd). The water droplet contact angle measured will be 7°. The low water droplet contact angle is indicative of the hydrophilic property of the coating.

Water resistance and Durability: The hydrophilic coating is subjected to a rubbing treatment with sponge in 10 reciprocations in water while applying a load of 1 kg. The amount of residual film is calculated from a change of weight before and after the rubbing treatment. The weight of the film after the rubbing treatment will be 97% of the initial weight.

Weather resistance: The hydrophilic coating is exposed in a chamber to a xenon arc lamp that is calibrated to mimic the sun spectral characteristics (Atlas Sun Test). The exposure is performed for 500 hours and evaluated with respect to hydrophilicity, water resistance and durability. The hydrophilic coating will exhibit the same properties before and after the exposure.

### Example 9

### A Paint with a Conventional Surfactant

About 15 grams of ethoxylated nonylphenol is mixed with 40 grams of TiO₂, 2 grams of thickener (hydroxyethyl cellulose), 150 grams of solvent (water), 70 grams of binder (methyl methacrylate), 0.3 grams of coalescing agent (2,2,4-trimethyl-1,3-pentanediolmono(2-methylpropanoate)), and 0.05 grams of bactericide. The components are mixed under high shear for 30 minutes.

### Example 10

### Evaluation of Hydrophilic Property of a Paint with a Conventional Surfactant

The coating preparation of Example 9 is coated on a glass surface and dried at room temperature. The surface free energy and the water droplet contact angle of the hydrophilic coating are measured as follows. A Zisman plotting method is employed for measuring the surface free energy. The surface tension of various concentrations of the aqueous solution of magnesium chloride is plotted along the X-axis, and the contact angle in terms of cos θ is plotted along the Y-axis. A graph with a linear relationship between the two is obtained. The graph is extrapolated such that the surface tension at contact angle 0° is measured and is defined as the surface free energy of the coated glass surface. The surface free energy of the glass surface measured will be less than 82 milliNewton/meter, indicating that the coating preparation of Example 9 is less hydrophilic than the coating preparation of Example 7.

The water droplet contact angle in air is measured by using DropMaster 500 (Kyowa Interface Science Co., Ltd). The water droplet contact angle measured will be higher than 7°, indicating that the coating preparation of Example 9 is less hydrophilic than the coating preparation of Example 7.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A compound of the formula I: 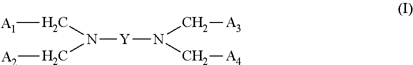
wherein
A₁ is —N(CH₂—CH₂—OH)₂, —O—C(═O)—(Z)ₙ—CH₃, —N(—CH₃)—(Z)ₙ—CH₃, —O—C(═O)—CH₂—CH₂—COOH, —O—C(═O)—CH₂—COOH, —O—SO₃H, or —O—PO₃H₂, or salts thereof, where each n is, independently, an integer from 1 to 25, and where Z is alkylene, arylene, alkenylene, alkynylene, acrylene, styrylene, or any combination thereof;
A₂ is —N(CH₂—CH₂—OH)₂, —O—C(═O)—(Z)ₚ—CH₃, —N(—CH₃)—(Z)ₚ—CH₃, —O—C(═O)—CH₂—CH₂—COOH, —O—C(═O)—CH₂—COOH, —O—SO₃H, or —O—PO₃H₂, or salts thereof, where each p is, independently, an integer from 1 to 25, and where Z is alkylene, arylene, alkenylene, alkynylene, acrylene, styrylene, or any combination thereof;
A₃ is —N(CH₂—CH₂—OH)₂, —O—C(═O)—(Z)_{q}—CH₃, —N(—CH₃)—(Z)_{q}—CH₃, —O—C(═O)—CH₂—CH₂—COOH, —O—C(═O)—CH₂—COOH, —O—SO₃H, or —O—PO₃H₂, or salts thereof, where each q is, independently, an integer from 1 to 25, and where Z is alkylene, arylene, alkenylene, alkynylene, acrylene, styrylene, or any combination thereof;
A₄ is —N(CH₂—CH₂—OH)₂, —O—C(═O)—(Z)ᵣ—CH₃, —N(—CH₃)—(Z)ᵣ—CH₃, —O—C(═O)—CH₂—CH₂—COOH, —O—C(═O)—CH₂—COOH, —O—SO₃H, or —O—PO₃H₂, or salts thereof, where each r is, independently, an integer from 1 to 25, and where Z is alkylene, arylene, alkenylene, alkynylene, acrylene, styrylene, or any combination thereof; and
Y is —C(═O)—, —CH₂—CH₂—, —CH₂—(CH₂)ₖ—CH₂—, —C(═O)—NH—C(═O)—, or polyurea, where k is an integer from 1 to 10.

2. The compound of claim 1, wherein any one of A₁, A₂, A₃ and A₄ is independently selected from —N(CH₂—CH₂—OH)₂, —O—C(═O)—(Z)ₙ—CH₃, —O—C(═O)—CH₂—CH₂—COOH, or —O—SO₃H, or salts thereof.

3.-5. (canceled)

6. The compound of claim 1, wherein:
A1 is —N(—CH3)-(CH2)20-CH3 or —O—PO3H2;
A2 is —N(—CH3)-(CH2)20-CH3 or —O—PO3H2;
A3 is —N(—CH3)-(CH2)20-CH3 or —O—PO3H2;
A4 is —N(—CH3)-(CH2)20-CH3 or —O—PO3H2; and
Y is —C(═O)—or —CH2-(CH2)k-CH2-.

7. The compound of claim 1, wherein
A₁ is —O—C(═O)—(CH₂)₂₀—CH₃,
A₂ is —O—C(═O)—(CH₂)₂₀—CH₃,
A₃ is —N(CH₂—CH₂—OH)₂,
A₄ is —N(CH₂—CH₂—OH)₂, and
Y is —C(═O)—NH—C(═O)—.

8. The compound of claim 1, wherein
A₁ is —N(—CH₃)—(CH₂)₂₀—CH₃,
A₂ is —N(—CH₃)—(CH₂)₂₀—CH₃,
A₃ is —N(CH₂—CH₂—OH)₂,
A₄ is —N(CH₂—CH₂—OH)₂, and
Y is —C(═O)—NH—C(═O)—.

9. The compound of claim 1, wherein
A₁ is —N(—CH₃)—(CH₂)₂₀—CH₃,
A₂ is —N(—CH₃)—(CH₂)₂₀—CH₃,
A₃ is —O—C(═O)—CH₂—CH₂—COO⁻.Na⁺,
A₄ is —O—C(═O)—CH₂—CH₂—COO⁻.Na⁺, and
Y is —C(═O)—NH—C(═O)—.

10. The compound of claim 1, wherein
A₁ is —N(—CH₃)—(CH₂)₂₀—CH₃,
A₂ is —N(—CH₃)—(CH₂)₂₀—CH₃,
A₃ is —O—SO⁻₃.Na⁺,
A₄ is —O—SO⁻₂.Na⁺, and
Y is —C(═O)—NH—C(═O)—.

11. The compound of claim 1, wherein
A₁ is —N(—CH₃)—(CH₂)₂₀—CH₃,
A₂ is —N(—CH₃)—(CH₂)₂₀—CH₃,
A₃ is —O—PO₃²⁻.(Na⁺)₂,
A₄ is —O—PO₃²⁻.(Na⁺)₂, and
Y is —C(═O)—NH—C(═O)—.

12. A method of making a surfactant, the method comprising:
contacting any one of urea, biuret, or alkylene diamine with formaldehyde to form a tetrahydroxy methyl compound;
contacting the tetrahydroxy methyl compound with any one of fatty acid, anhydride, acid chloride or N-methylamino compound to form a di-substituted intermediate compound; and
contacting the di-substituted intermediate compound with any one of an amine, succinic anhydride, chlorosulfonic acid, or chlorophosphoric acid to form the surfactant.

13. The method of claim 12, wherein contacting the tetrahydroxy methyl compound comprises contacting with a fatty acid anhydride having 5-25 carbon atoms comprising alkylene, arylene, alkenylene, alkynylene, acrylene, or styrylene groups, or any combination thereof.

14. The method of claim 12, wherein contacting the tetrahydroxy methyl compound comprises contacting a N-methylamino compound having 5-25 carbon atoms comprising alkylene, arylene, alkenylene, alkynylene, acrylene, or styrylene groups, or any combination thereof.

15. The method of claim 12, wherein contacting with the amine comprises contacting with trialkyl amine, monoethanol amine, diethanol amine or triethanol amine.

16. The method of claim 12, wherein contacting any one of urea, biuret, or alkylene diamine with the formaldehyde comprises contacting any one of urea, biuret, or alkylene diamine with formaldehyde in a molar ratio from about 1:2 to about 1:6 in the presence of a basic catalyst in a solution having a pH of about 8 to a pH of about 11.

17.-19. (canceled)

20. The method of claim 12, wherein contacting the tetrahydroxy methyl compound with any one of fatty acid, anhydride, acid chloride or the N-methylamino compound comprises contacting the tetrahydroxy methyl compound with any one of fatty acid, anhydride, acid chloride or the N-methylamino compound in a molar ratio from about 1:2 to about 1:4.

21.-22. (canceled)

23. The method of claim 12, wherein contacting the di-substituted intermediate compound with any one of the amine, succinic anhydride, chlorosulfonic acid, or chlorophosphoric acid comprises contacting the di-substituted intermediate compound dissolved in a solvent with any one of the amine, succinic anhydride, chlorosulfonic acid, or chlorophosphoric acid in a molar ratio of about 1:3 to about 1:6.

24.-26. (canceled)

27. The method of claim 12, further comprising contacting the surfactant with a molar excess of a hydroxide to form a salt selected from NaOH, KOH, NH₄OH Mg(OH)₂, Ca(OH)₂, or any combination thereof.

28. (canceled)

29. A hydrophilic coating composition comprising:
a surfactant of the formula I 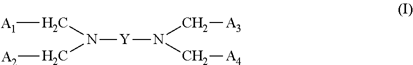
wherein
A₁ is —N(CH₂—CH₂—OH)₂, —O—C(═O)—(Z)ₙ—CH₃, —N(—CH₃)—(Z)ₙ—CH₃, —O—C(═O)—CH₂—CH₂—COOH, —O—C(═O)—CH₂—COOH, —O—SO₃H, or —O—PO₃H₂, or salts thereof, where each n is, independently, an integer from 1 to 25, and where Z is alkylene, arylene, alkenylene, alkynylene, acrylene, styrylene, or any combination thereof;
A₂ is —N(CH₂—CH₂—OH)₂, —O—C(═O)—(Z)ₚ—CH₃, —N(—CH₃)—(Z)ₚ—CH₃, —O—C(═O)—CH₂—CH₂—COOH, —O—C(═O)—CH₂—COOH, —O—SO₃H, or —O—PO₃H₂, or salts thereof, where each p is, independently, an integer from 1 to 25, and where Z is alkylene, arylene, alkenylene, alkynylene, acrylene, styrylene, or any combination thereof;
A₃ is —N(CH₂—CH₂—OH)₂, —O—C(═O)—(Z)_{q}—CH₃, —N(—CH₃)—(Z)_{q}—CH₃, —O—C(═O)—CH₂—CH₂—COOH, —O—C(═O)—CH₂—COOH, —O—SO₃H, or —O—PO₃H₂, or salts thereof, where each q is, independently, an integer from 1 to 25, and where Z is alkylene, arylene, alkenylene, alkynylene, acrylene, styrylene, or any combination thereof;
A₄ is —N(CH₂—CH₂—OH)₂, —O—C(═O)—(Z)ᵣ—CH₃, —N(—CH₃)—(Z)ᵣ—CH₃, —O—C(═O)—CH₂—CH₂—COOH, —O—C(═O)—CH₂—COOH, —O—SO₃H, or —O—PO₃H₂, or salts thereof, where each r is, independently, an integer from 1 to 25, and where Z is alkylene, arylene, alkenylene, alkynylene, acrylene, styrylene, or any combination thereof; and
Y is —C(═O)—, —CH₂—CH₂—, —CH₂—(CH₂)ₖ—CH₂—, —C(═O)—NH—C(═O)—, or polyurea, where k is an integer from 1 to 10.

30. The coating composition of claim 29, further comprising a binder, a solvent, a pigment, a rheology modifier, a plasticizer, or any combination thereof.

31. The coating composition of claim 29, wherein the surfactant is covalently attached to a binder.

32. The coating composition of claim 29, wherein the surfactant comprises a plurality of surfactants cross-linked to each other.

33. (canceled)

34. The coating composition of claim 29, further comprising a binder comprising a polymer of alkylacrylate, alkyl methacrylate, allyl methacrylate, acrylic acid, methacrylic acid, acrylamide, 2-hydroxyethyl methacrylate, 2-hydroxypropyl methacrylate, thioethyl methacrylate, vinyl methacrylate, vinyl benzene, 2-hydroxyethyl acrylate, butyl acrylate, 2-ethylhexyl acrylate, vinyltrimethoxysilane, vinyltriethoxysilane, vinyl formate, vinyl acetate, vinyl propionate, vinyl butyrate, vinyl hexanoate, vinyltoluene, α-methyl styrene, chlorostyrene, or styrenesulfonic acid, or a copolymer of any of the foregoing, or any combination thereof.

35. The coating composition of claim 29, wherein the composition is a latex emulsion, aqueous solution, non-aqueous solution, or a powder.

36.-46. (canceled)
